(19) United States
(12) Patent Application Publication
(10) Pub. No.: US 2004/0014041 A1
(43) Pub. Date: Jan. 22, 2004

(54) Method and system of evolutionary phenogenetic engineering

(76) Inventor: Michael Conroy Allan, Toronto (CA)

Correspondence Address: Michael Allan, c/o Margaret Allan, 40 West Avenue South, Apartment 302, Hamilton, ON, L8N 2S1, CA

(21) Appl. No.: 10/192,432

(22) Filed: Jul. 11, 2002

(51) Int. Cl. C12Q 1/68; G06G 7/48; G06G 7/58; G06F 19/00; G01N 33/48; G01N 33/50

(52) U.S. Cl. 435/6; 702/20; 703/11

(57) ABSTRACT

A method and system to facilitate the design of artifacts by a process of participative refinement; this process being analogous to natural selection as it operates in biological populations. In this invention, designs are encoded as genetic data structures. Participants publish the encoded designs through a communication network to form a population of variant genotypes. The genotypes are subject to human guided mutation and recombination, resulting in the progressive refinement of the designs.

### BACKGROUND OF THE INVENTION

The invention is comparable to methods of evolutionary and participative design from the fields of biological engineering, evolutionary computation, cultural anthropology, and collaborative design.

FIG. 1 provides a summary of the comparison. The present invention 119 and oral recomposition 125 are distinguished as the only methods that employ man as both the agent of variation 120 and the agent of selection 121, while acting directly at the level 122 of the gene.

### Biological Engineering

Comparable methods from the field of biological engineering are artificial selection, genetic engineering, and reverse genetic engineering.

Artificial selection 102 has long been applied to the biological engineering of agricultural and domestic organisms. The process is similar to Darwinian natural selection 100, except that the agent of selection 104 is man. Variations still arise through mutation and sexual recombination 103, as in nature. Breeding stock is still selected from among whole individuals 105, as in nature. The ultimate effect is at the level of the gene (Dawkins, 1982), but there is no direct selection at that level, neither by the artificial breeder nor by nature.

Direct selection at the genetic level 108 occurs in genetic engineering 106. Particular genes are selected for their phenotypic traits, and injected into the genotype of a target individual, often from a different species. The source of the genes is still natural 107. Furthermore, the procedure is a single step, and not in itself evolutionary.

In reverse genetic engineering 110 the new genetic material is fabricated 111 rather than selected from nature. The procedure is therefore non-selective. It is also non-evolutionary.

In FIG. 1, both genetic engineering 106 and reverse genetic engineering 110 are shown in italics, to indicate that they are not evolutionary methods in themselves, but isolated, one-time procedures.

### Evolutionary Computation

In methods of evolutionary computation 115, the machine acts in place of nature. In most methods, the machine acts as both the agent of variation 112, and the agent of selection 113. In other methods, most notably in evolutionary art, the agent of selection 117 may instead be man (Bentley, 1999). In all methods, however, the machine remains the agent of variation 112 116. It typically generates mutations and recombinations at random. In all methods, direct selection is at the level 114 118 of the whole individual.

Reverse genetic engineering 110 is a procedure not only of biological engineering 109, as previously explained, but also of evolutionary computation 115. Bentley (1999) describes ‘knowledge seeding’ for evolutionary computation as a means of jump starting a long-term evolutionary process, by injecting the start-up population with proven genetic material. Where the injected material is man-made, this would be a form of reverse genetic engineering. As such, it would be both non-selective and non-evolutionary in itself.

### Cultural Anthropology

Comparable methods from the field of cultural anthropology are memetics and oral recomposition.

In memetics, it is supposed that cultural artifacts evolve by a process similar to Darwinian natural selection. Variant artifacts compete for survival in society. Each variation is based on the mutation and re-assortment of constituent memes.

Memes are analogous to genes, but with numerous distinctions. They exist in the dynamic memory of people; rather than in static chromosomes. Their encoding structure is unknown. They do not have identifiable loci or alleles. Their copying fidelity is low. They can re-associate by blending instead of by particulate recombination. And their genotypes are subject to causal feedback from their phenotypes. These distinctions detract from the usefulness of the genetic analogy (Dawkins, 1982). Memetics is therefore a weak theory, and has no practical applications.

A sounder mechanism of cultural evolution is oral recomposition. It is based on the evidence from Milman Parry's (1928) study of Homeric song that the ancient epics The Iliad and The Odyssey were composed largely of recurring epithets and stock phrases. Each such ‘prefabricated part’ (Ong, 1982) had been selected from a limited palette of choices, to fit within the metre of the surrounding verse. Ong traces the necessity of such composition to the limitations of the oral culture of pre-literate Greece in which the epics took shape. Only formula and cliche could have taken hold in cultural memory, to survive intact from generation to generation. Oral recomposition is therefore an evolutionary process. The Iliad and The Odyssey were finally written down in the sixth century B.C., after evolving for perhaps a thousand years in human memory (Nagy, 1992), prior to the invention of the Greek alphabet. Their achievement was such that, unchanged some 2,500 years later, they still mark the heights of Western literature. In explaining this success, it may help to consider that the same ‘prefabricated parts’ which purchased holds for mere survival in human memory, might also have purchased holds for a process of evolution. Within the milieu that Parry and Ong describe, oral poetic forms and fragments would, in fact, be a firmer substrate for selection than memes. They were copied faithfully and systematically so that, although restricted to memory like memes, they nevertheless proved quite durable.

Their durability was confined to their small scale, however, because at a larger scale the epic as a whole was never memorized verbatim—instead it was recomposed from telling to telling (Ong, 1982). This resulted in a series of unique combinations from a limited pool of conservative parts. And this, essentially, is how genotypes recombine from generation to generation in sexual populations. So it is not difficult to see how evolution could have taken hold; allowing a line of Iliads, for example, to evolve beyond the creative talents of any single poet.

Although this conclusion is only speculative, oral recomposition 125 is shown in FIG. 1 as a method of evolution by direct genetic selection 126. This would make it similar to the present invention 119. However, it differs in being non-deliberate, and non-purposeful. It would also be much slower in action, because its pace would be tied to the cycle of individual recitations. Each recitation would need to be heard in its entirety, before anyone in the audience could be impressed with its genetic innovations. The speed of evolution would thus be limited by the speed and frequency of recitation, and the range of travelling singers.

### Collaborative Design

Hirschberg and Wenz (2000) describe an experiment in the field of collaborative design called Phase (x). An evolutionary method, it divides a design effort into several phases. Between every two successive phases there is a round of selection in which the designer examines the work of others, and chooses a single design with which to continue on with in the second phase. Although the authors describe this as ‘memetic engineering’, the analogy is misleading, because memes are not the units of selection in Phase (x), as genes are in genetic engineering. The actual units 127 of selection are whole individual designs or design phases, rather than their constituent memes.

Phase (x) is distinctive as an evolutionary process in having a definite end, which is marked by its final design phase. Evolutionary processes are usually more open-ended.

Other research in this field rarely touches on evolutionary processes. The common assumption is that of a team of designers focused on the completion of a single work (Kvan, 2000). Open, competitive and potentially undirected processes are not an obvious fit. Informal evolutionary processes do appear, however, in common design practice. Consider automotive design as an example. The final products are assembled from parts obtained on the open market. There is a degree of variation among the parts designs of competing vendors; and by selecting from among these, automotive firms encourage the adoption of the best innovations. Improvement in parts, as well as in assembly, is thus driven by professional competition among technicians and engineers, and by commercial competition among firms. The evolution of automotive design inches forward in response, from model to model. This is a slow and informal process, operating similarly in a wide range of industries.

What is needed to accelerate this process is to free it from the cycle of the production line, and to purposely apply it to the design of a single model, prior to manufacture. A population of prototypes could then evolve in the design line, from compositions of virtual parts and virtual assemblies chosen from among the offerings of competing designers. Evolution, thus compressed in time, could drive the design of a single product prior to manufacture. A method to enable such a design process has not previously been reported.

Any design process which is open to some degree is likely to become competitive, and might be viewed as an evolutionary process in a Darwinian sense. Consider open source software development. This process is currently being applied to a number of different projects, the best known of which are the Linux operating system, and the Apache web server.

Contributors compete to deliver designs to each project in the form of source code modules. As a process, however, open source is largely ad hoc-really ‘no process at all’ (Raymond, 1997). For example, it defines no formal procedure for competitive selection among contributions, or among whole assemblies of the product. Competition is not especially encouraged, particularly at the level of the whole assembly. A single authority compiles each official release. The design process is thus evolutionary only in that it progresses, i.e. toward a more functional version of the software, with incremental improvements from release to release. But it is not evolutionary in the Darwinian sense; not comparable with the struggle that occurs in nature. It is unlikely, for example, that a large population of Linux variants would arise; or that Apache would split into several competing lineages, some of which would diverge into applications other than web serving. Undirected outcomes such as this are not the intent of open source projects. Open source is neither formally nor substantially an evolutionary process in this sense.

Methods of open source design do not address the genetic encoding of source modules; the maintenance of a population of variant encodings; and the recombination of genes-all of which are necessary to an efficient process of evolution. There is no easy way, for example, to search through a population of modules, and select among variants with respect to a particular portion of code. There are too many non-standard methods of publishing modules, so that a list of all variants would take too much effort to compile. Once compiled, it would be necessary to exhaustively read through each variant module, in order to find and isolate the particular portion of code; then to compare among all variants with respect to that portion. If, instead, the logical structure of the code could be broken up at a smaller scale, into an arrangement of uniquely identified genes, then it would be possible to implement an efficient procedure of recombination. The missing key is the encoding of genetic identity within the source code.

At a higher level of the open source process, where modules are assembled into working software applications (or in similar processes of component assembly from other fields, wherever the internal design of components is open) the process comes closer to formal genetics. At this level, modules may be viewed as large ‘genes’. They are amenable to human guided ‘mutation’, to produce a pool of variants, because the source code is open. The choice of a particular combination of these variants defines a ‘genotype’, which may in turn be compiled into a working application.

Again, however, this process lacks an efficient procedure of recombination. Whole combinations—i.e. genotypes—are not nearly as open to design inspection as are the component modules. After initial combination, for example by an independent Linux user, the new genotype is usually stored in private on the users machine. Nobody else can easily inspect this genotype in order to select genes for recombination into their own variant of the genotype. The population of whole individuals is thus largely invisible, and effectively non-existent. The module-genes themselves may get published, but usually separate from genotypes; for example on various web sites that offer a catalogue of Linux modules. These may be combined, and then recombined privately; but an efficient procedure of public recombination is lacking.

Public distributions do exist, e.g. for Linux, but their constituent genotypes are not also published. Several distributions would have to be purchased; opened up to inspection; and recombined from this restricted set of choices. The result, however, would not subsequently be republished. Without a formally defined genetic code and without a method of publishing and viewing a population of variants-recombination remains difficult, and the overall process of evolution stalls.

### BRIEF SUMMARY OF THE INVENTION

The invention provides a participative method for facilitating the evolutionary design of a species of artifact. The species has a population consisting of individuals of the species, each individual is encoded by an instance of a genotype, each genotype is formed according to a phenogenetic grammar, each individual and its instance of a genotype are associated with a participant from a community of participants, and the community is inter-linked by a data network. The method includes selecting an instance of a genotype associated with a participant under direction of said participant; applying an alteration procedure to the instance of the genotype under direction of the participant, wherein the alteration procedure is either a mutation or a recombination; and publishing via the network the result of the alteration procedure for display to participants of the community.

A mutation may delete a gene from the instance of the genotype, alter the content of a gene, add a gene to the instance of the genotype, rearrange a gene with respect to other genes within the instance of the genotype, whereby the location of the gene is modified within the instance of the genotype, or introduce a pre-existing gene or genetic fragment from the population, other populations, or other species, into the instance of the genotype. Every mutation creates a new allele. In a recombination, on the other hand, the participant associated with the genotype selects one of these alleles, an instance of which is published via the network, being an allele of a gene; replicates the instance of the allele to create a new instance of the allele; and substitutes the new instance of the allele into the instance of the genotype, wherein the new instance of the allele replaces an instance of a different allele of the same gene.

The invention also relates to a system for facilitating the evolutionary design of a species of artifact, the species having a population consisting of individuals of the species, each individual encoded by an instance of a genotype, each genotype formed according to a phenogenetic grammar, each individual and its instance of a genotype associated with a participant from a community of participants, the system including software and hardware elements forming a network of computers. The software and hardware elements include a first element for selecting an instance of a genotype associated with a participant under direction of said participant; a second element for applying an alteration procedure to the instance of the genotype under direction of the participant, wherein the alteration procedure comprises at least one procedure selected from the group consisting of mutations and recombinations; and a third element for publishing the result of the alteration procedure, whereby the result may be examined by participants of the community. The network may be based on a client-server model, or a peer-to-peer model.

### BRIEF DESCRIPTION OF THE SEVERAL VIEWS OF THE DRAWING

FIG. 1 is a table comparing different methods of evolutionary design, across a range of fields. The present invention 119 is distinguished, along with oral recomposition 125, in employing man as both the agent of variation 120 and the agent of selection 121, while acting directly at the level 122 of the gene.

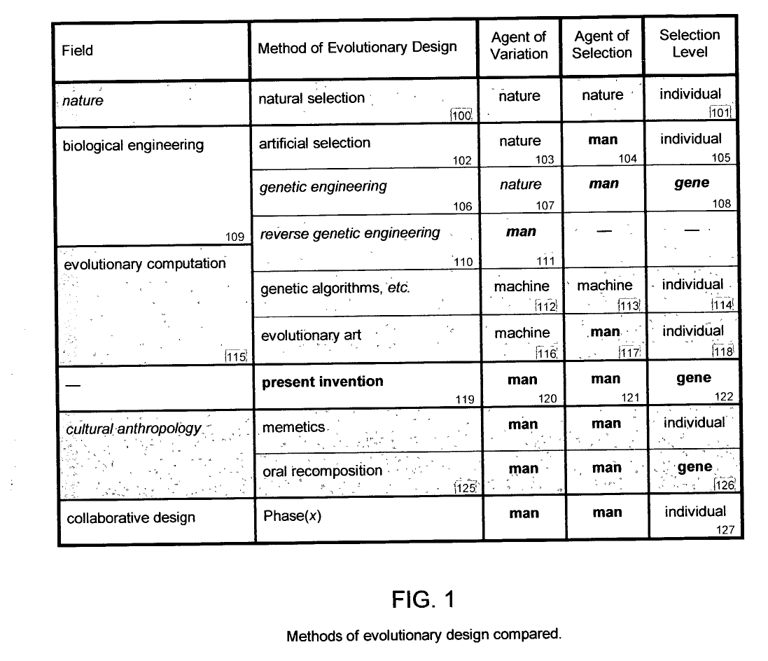

FIG. 2 shows a summary 200 of the constituent genotype of an example of an individual artifact, and its association 201 with a specific participant in the method, identified as ‘A’.

FIG. 3 shows an example of population growth by individual replication of an original instance of a genotype 300, resulting in two new instances 301 302.

FIG. 4 shows an example of several procedures of mutation, introducing genetic diversity to the population in the form of new alleles 403 404 405 406.

FIG. 5 shows an example of several procedures of recombination 503 504 505; and a single mutation 506.

FIG. 6 is a UML deployment diagram of an example of a client-server configuration of the system, also showing components 602 604 of the population server 600.

FIG. 7 is a UML deployment diagram of an example of a component based workstation 700, also showing network connections 706 for a peer-to-peer configuration of the system.

FIG. 8 shows an example of a genotype-to-phenotype mapping 800 from a literary embodiment. This particular example uses an XML based phenogenetic grammar. It shows the genetic encoding 801 and phenotypic expression 802 of one individual from the subspecies of Ralph Waldo Emerson's poem Brahma.

### DETAILED DESCRIPTION OF THE INVENTION

Evolutionary phenogenetic engineering (“EPE”) is a multi-person, participatory process for the creation and rapid refinement of design intensive artifacts. It works like natural selection within biological populations, using a similar mechanism of genetic variation and differential reproduction. The technical innovation is the acceleration of the design process, first by unleashing it from the slower pace of somatic replication in the production line, and second by propelling it forward in the design line, by applying direct human guidance at the genetic level.

Instead of the molecular genetics and physical spatial populations of an organic process, EPE relies on data structural phenogenetic codes and data communication networks. EPE also differs from natural selection, artificial selection, and evolutionary computation in that variation and selection are under direct human guidance, and both operate at the genetic level. This is the ‘engineering’ aspect of the process. It differs from the typical method of introducing variation at the genetic level by random mutation, while selecting by reproduction at the individual level.

### Phenogenetic Grammar

Each embodiment of the method requires the definition of a phenogenetic grammar. A phenogenetic grammar is a fundamental set of rules for encoding genetic information within EPE; one that:

1) specifies how to encode data within a gene

2) specifies how to compose a genotype from multiple genes

3) specifies how to express an individual artifact's phenotype from its genotype

4) allows for the encoding of a wide range of genotypes, ideally expressing all conceivable phenotypes of the species

5) specifies how to replicate individual artifacts

6) specifies how to mutate genotypes

7) specifies how to recombine genotypes

The above requirements are also met by the natural genetic grammar of biological organisms, and the artificial codes employed in evolutionary computation.

Furthermore, EPE requires a grammar that is phenogenetic, i.e. one that:

8) allows for the mapping of genotypes to phenotypes in such a way as to facilitate human guided mutation and selection of alleles, according to their phenotypic traits

9) specifies how to encode the unique identity of genes And, optionally, that:

10) specifies how to encode the authorship of alleles

A gene is a data structure which may be combined with other genes in order to form the genome of a species. Specific variants of a gene are termed alleles. A specific variant of a genome, sufficient to express the phenotype of an individual of the species, is termed a genotype. A genotype is defined by a particular combination of alleles. Genes, alleles, genomes and genotypes are constructed according to the phenogenetic grammar.

For example, if the embodiment targets the field of literature, where each subspecies is a particular novel, poem, essay, treatise or other literary work, then a suitable phenogenetic grammar would be one that is based on text; preferably in a data structural format such as ASCII, XML or SGML. A gene could then be a sequence of text elements, each comprising a machine readable line, phrase or sentence. Its unique identity could be encoded as a serial number in associated mark-up. The genotype might be defined as a linear sequence of genes. Replication of individuals might be accomplished by ordinary data copying of the genotype. Expression might involve loading the genotype into a suitable text viewer, web browser, or other document viewer, which would parse and format the text, transcribing it and presenting it to the reader as a fully formed individual of the species: a readable novel, poem, essay, etc.

Or, if the field is applied molecular chemistry, then the phenogenetic grammar might follow the periodic table of elements, defining a gene as a virtual atom encoded by atomic number; and the genotype as a 3-dimensional pattern of chemical bonding. Expression of an individual molecule might involve analyzing its predicted properties using automated tools; or physically creating it in the laboratory.

Or, if the field is software engineering, then the phenogenetic grammar might follow the hierarchical and sequential structure of source code, defining a gene variously as a package, module, declaration or statement, depending on its level in the hierarchy. Expression of an individual might involve visual inspection of the readable source code, and/or its compilation into executable form.

Or, if the field is genetic engineering, then the phenogenetic grammar might follow the actual genetic grammar of the target organism, recorded as a data structural representation of a polynucleotide sequence. The genotype might then be a set of such representations of polynucleotide sequences. Phenotypic expression might involve the use of automated tools to predict the transcription sequence and resulting mix of protein end products; or, ultimately, the insertion of the equivalent actual sequences into the target organism, altering its own larger phenotype.

The phenogenetic grammar must be defined in such a way as to allow genotypes to be mutated in order to create new alleles; and to allow instances of different alleles to be recombined among genotypes. These requirements are further elaborated in the sections below, where the process of EPE itself is described.

The phenogenetic grammar must have a genotype-to-phenotype mapping that facilitates human guided mutation and selection of alleles according to phenotypic traits. This is a critical, because selection occurs during recombination in EPE and must therefore be conducted at the genetic level. The alleles evaluated during selection must be the products of human guided mutation. The phenotypic effects of alleles must be made apparent, at least in comparison with other alleles. One way to simplify these tasks is to keep the genotype-to-phenotype mapping itself simple.

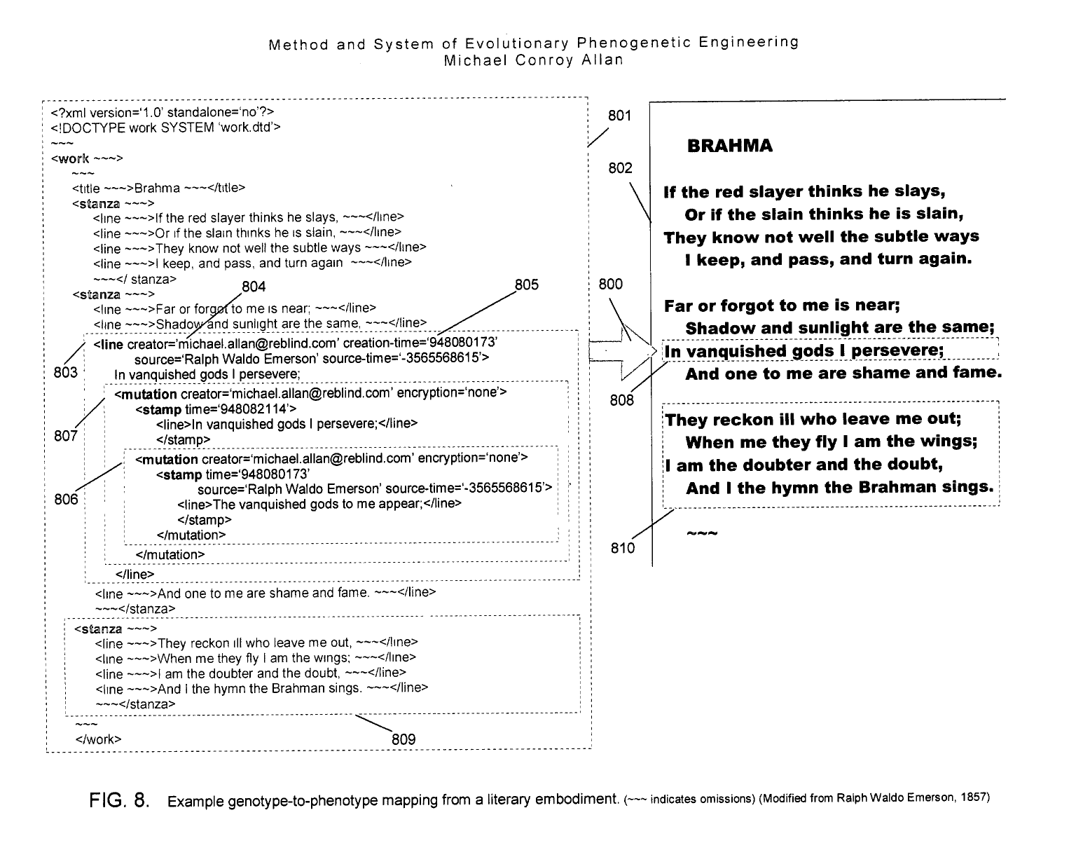

FIG. 8 shows an example genotype-to-phenotype mapping 800. Note the logical correspondence between the form of the genotype 801 and its phenotype 802. Each line gene (e.g. 803) corresponds to a line (i.e. 808) of the poem; each stanza gene (e.g. 809) to a stanza (i.e. 810). In this particular example, drawn from a literary embodiment, the superficial typography and physical form of the phenotype 802 (generated, for example, by a particular configuration of XML browser/editor/printer) is independent of the genotype 801; but its essential poetic form is entirely defined by the genotype 801.

Only excerpts of the genotype 801 and phenotype 802 are shown in FIG. 8. The omitted portions of each are indicated by ellipsis symbols (˜˜˜).

To facilitate searching for alleles on the network, the phenogenetic grammar must allow the unique identity of genes to be encoded. Thus even after internal mutation of a gene, or its relocation within a genotype, every one of its alleles may still retain its former identity as a variant of that particular gene. In this way, regardless of how they are altered or relocated within an instance of a genotype, the genetic identity of genes and alleles may remain independent of their data content, and their location.

In the example literary grammar, genes are uniquely identified by the combined attributes of ‘creator’ and ‘creation-time’. For example, in the particular genotype shown 801, the third line gene 803 in the second stanza was created by the participant identified 804 as michael.allan@reblind.com, at a particular moment 805 in the year 2000—the line having been copied from Ralph Waldo Emerson's original publication of 1857.

In this particular embodiment, each time stamp (e.g. 805) is encoded as a positive or negative offset from a standard base time (UTC), and expressed in units of seconds.

Genetic identity need not always be explicitly encoded. If the phenogenetic grammar provides additional information for each allele, such as an alteration history, and if the embodiment automates a reasonably accurate and efficient method of determining the original identity from this additional information, then the requirement of genetic identity is sufficiently met.

The phenogenetic grammar must usually record the authorship of individual mutations. Typically this will serve to establish a legal ownership over the mutation itself; and a share in the ownership of the resulting allele, and any genotype that contains an instance of it, and any artifact expressed from such a genotype.

For example, in the genotype 801 of FIG. 8, mutation elements 806 807 encode the creator, time and content of the original mutation 806 that created the gene 803, and of a subsequent mutation 807. As new mutations occur, they will be encoded into new elements that ‘wrap’ the previous ones, thus encoding the entire history of mutations in a sequence that can later be unfolded to any depth.

Authorship of recombinations may also be encoded; for similar purposes, and by similar means. Collective and independent declarations and contracts of ownership and licensing may also be encoded.

The suitable definition of a phenogenetic grammar is expected to vary from embodiment to embodiment according on the characteristics of the target species, and the preferences of the implementer. Except for the broad requirements defined here, and in the sections below, the details of the actual definition chosen are not critical to the process.

### Evolutionary Phenogenetic Engineering

EPE begins with one or more individual artifacts of the species. At its most basic, the process begins with just a single artifact, intended as a work in progress.

For example, in a lexicographical embodiment, the initial artifact might be a newly created dictionary with a single entry, as follows:

evolution i:vg [symbol] ′lu•∫( [symbol] )n gradual change.

The designer then takes this initial artifact, and reverse transcribes it into the phenogenetic grammar of the embodiment. This step encodes the initial artifact into a genotype formed according to the phenogenetic grammar. The encoding might be done by hand, but typically an automated tool will be used instead. Typically the tool will belong to a suite of EPE tools in use at the designer's node or workstation.

To continue the example, assume a particular phenogenetic grammar based on XML. The reverse transcription of the initial dictionary might be encoded as follows:

<?xml version=“1.0” standalone=“no”?>
<!DOCTYPE lexicon SYSTEM “lexicon.dtd”>
<lexicon id=“a109”>

<entry id=“a1”>

<word id=“a1”>evolution</word>
<pronunciation id=“a1”>i:v [symbol] ′lu:∫( [symbol] )n</pronunciation>
<meaning id=“a1”>

gradual change
</meaning>

</entry>

</lexicon>

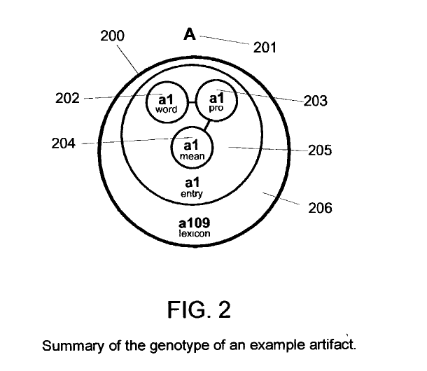

This defines a genotype, as summarized in FIG. 2. It is composed of 5 genes 202 203 204 205 206 in a sequential/hierarchical data structure 200: a <word>gene 202, followed by a <pronunciation>gene 203, followed by a <meaning>gene 204; nested in a single <entry>gene 205; nested in a single <lexicon>gene 206.

The genotype is then published so that other participants may become aware of it, and evaluate it. EPE is necessarily a participative process, combining the efforts of several people-ideally many. These people are equivalently termed ‘participants’, or ‘designers’, or ‘phenogenetic engineers’. Publication of a new instance of a genotype encoded from an entirely original artifact results in a population of size one. The single member of the population is the artifact itself, as originally created.

To continue the example, let the original lexicographer, who we will identify 201 as participant A, publish the corresponding genotype 200. This will result in a population consisting of a single individual, encoded by its genotype 200, and associated 201 with participant A.

Participants may recover any artifact from its genotype, as published, using the procedure of expression. In expression, the genotype-to-phenotype mapping of the phenogenetic grammar are applied, and the resulting data is then made intelligible to human participants as a comprehensible instance of the artifact. Therefore a 1:1 correspondence exists between a published genotype and the expressed artifact it encodes.

Full expression of an entire individual is not always required. Instead, a single allele or other fragment of its genotype may be expressed on its own, and evaluated by the phenogenetic engineer. Partial expression is thus employed, for example, during the procedure of recombination (as described further below). In that procedure, the partially expressed phenotype of a source individual, whose full phenotype likely remains unknown, is evaluated within the context of a target individual whose full phenotype is already familiar to the engineer, being the individual associated with the engineer.

The process scales up by individual replication, which enlarges the population. This step begins with the copying of a genotype, and its transmission to a different location—e.g. another node on the network, or a different workspace on a single node—where a new participant is associated with it. Each replicated instance is eventually republished to create a new individual of the species. This will increase the size of the population. Initially each new individual will be a clone of the parent from which it was copied.

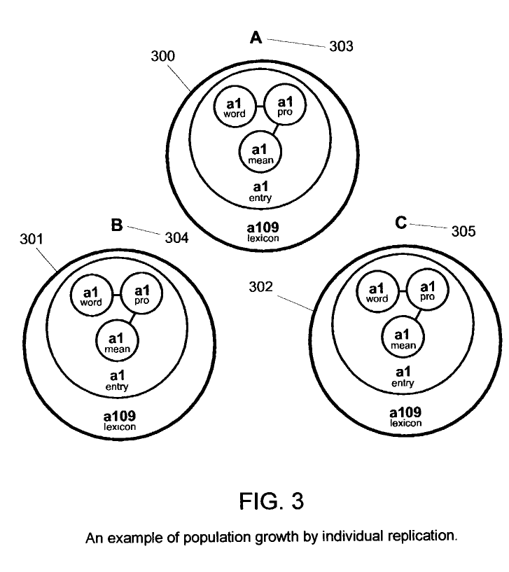

For example, as illustrated in FIG. 3, assume two new participants (designated 304 305 as B and C) are browsing the network. They discover the dictionary genotype 300 previously published by A. They copy it by individual replication. The result is a population of three individuals represented by three separate genotypes 300 301 302, all clones of each other; and

Each associated 303 304 305 with its own participant. Each newly replicated individual typically remains a clone until its genotype is altered by one or more procedures of mutation or recombination. Alterations occur at the discretion of the associated participant, at times of the participant's own choosing. Participants alter their own associated genotypes; not those of other participants.

Individual replication may typically be combined with these alteration procedures. Publication would thus be delayed until after alteration. The end result would be the same.

### Mutation

Mutation is any act of altering a genotype, except that of recombination. Mutation includes alteration to the content of a gene; deletion or addition of a gene; and other rearrangements of genes with respect to each other. EPE requires that different participants be allowed to effect mutations of their own devising. A series of cumulative mutations is one of the principal subprocesses of EPE.

For example, seeing the possibility of improving on A's original definition of ‘evolution’, B and C go to work separately, and alter it. Their alterations are automatically reverse transcribed as mutations, and encoded into their own separate genotypes 401 402. Here is the result for B's genotype 401:

<?xml version=“1.0” standalone=“no”?>
<!DOCTYPE lexicon SYSTEM “lexicon.dtd”>
<lexicon id=“a109”>

<entry id=“a1”>

<word id=“a1”>evolution</word>
<pronunciation id=“a1b1”>i:v [symbol] ′lu:∫( [symbol] )n. ′εv [symbol] lu:∫( [symbol] )n</pronunciation>
<meaning id=“a1”>

gradual change
</meaning>

</entry>

</lexicon>

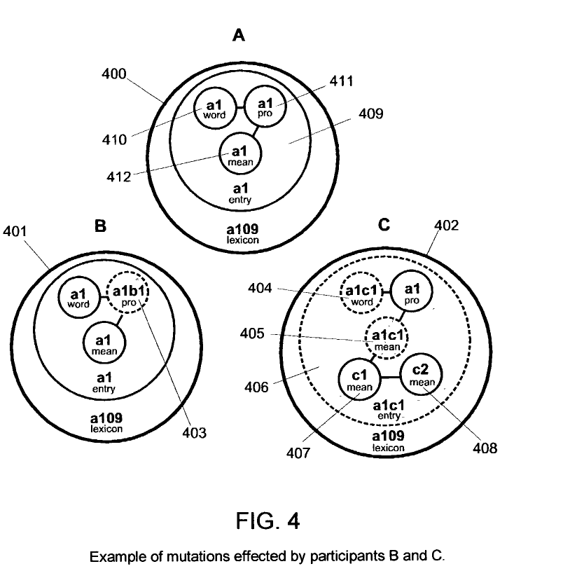

Thus B has added an alternate pronunciation, which is automatically reverse transcribed as a mutation 403 of <pronunciation>gene ‘a1’, as shown above, and as summarized in FIG. 4. (The gene is re-attributed as ‘a1b1’. For purposes of this illustrative example, a simplistic encoding scheme is used, in which an allele's genetic identity is appended by a history of its mutation and authorship, all concatenated into a single ‘id’ attribute.)

At the same time, working separately, C effects the following more substantial mutations 404 405 406, including the addition 406 of two entirely new <meaning>genes 407 408:

<?xml version=“1.0” standalone=“no”?>
<!DOCTYPE lexicon SYSTEM “lexicon.dtd”>
<lexicon id=“a109”>

<entry id=“a1c1”>

<word id=“a1c1”>evolution</word>
<pronunciation id=“a1”>i:v [symbol] lu:∫( [symbol] )n</pronunciation>
<meaning id=“a1c1”>

gradual progressive change
</meaning>

<meaning id=“c1”>

a process of development and origin of species from previous forms
</meaning>

<meaning id=“c2”>

the progression of events etc. in due course
<example>the evolution of the plot</example>
</meaning>

</entry>

</lexicon>

The mutant genotypes are then republished so that other participants may become aware of them, and evaluate them. Each mutation may create a unique alternative data content for a particular gene, or a unique alternative arrangement of genes, and each such unique alternative is termed an allele.

For purposes of definition, when the mutation is the deletion of a gene, then consider it as creating a null allele for that gene, where a null allele is an allele having no data content. When the mutation is the addition of a gene, then consider it as creating two new alleles, one of which is defined by the initial data content of the new gene, and the other being a null allele for that gene. When the mutation is some other rearrangement of genes with respect to each other, then it may be considered as a combination of additions and deletions.

In one class of embodiment, the semantics of what constitutes an allele may be simplified further by adopting a hierarchically complete phenogenetic grammar; one in which every collection of multiple genes is itself nested in some larger containing gene; a containing gene being simply a collection of smaller contained genes. This would allow any addition, deletion, or other rearrangement of genes, to be considered as a mutation altering the content of the containing gene or genes; resulting in the creation of a new allele of the containing gene or genes. The abstract concept of null alleles may, in this class of embodiment, be dispensed with.

The definition of an allele in EPE differs from biological terminology in that it extends beyond the individual to encompass the entire population. Alleles in biological terminology are typically considered only as alternative DNA content for corresponding genes among homologous chromosomes, all in a single individual. Thus for a diploid organism, with 2 homologues for each chromosome, there are 2 possible alleles for each gene. For a haploid organism, with only a single chromosome, the concept of an allele would not typically apply.

The concept of an allele is important to an understanding of biological meiosis, where the random assortment of homologous chromosomes, and random crossover among them, recombine alleles for the haploid gametes, and thence for the next generation. The number of different alleles thus available for recombination in a single generation of an individual is limited by the ploidy of the organism; e.g. 2 alleles for diploids, per gene, with at most 2 more from the opposite sex during fusion of gametes (subject to limitations of assortment) when a new diploid zygote is formed.

In EPE however, for any one generation, the entire population of genotypes is available as a source of genetic alternatives. In this sense the artifacts of EPE are hyperploids, whose chromosomal homologues extend out to the size of the population. Therefore, for purposes of EPE, each unique alternative in the population at large for a particular gene or collection of genes is termed an allele.

In summary, the population of artifacts in EPE consists of variant individuals possessing variant genotypes owing to different choices of alleles.

Assume that B and C have each completed their work, for the present. Separately they choose to publish the results, as illustrated in FIG. 4. The new variant genotypes 401 402 will replace the old clones 301 302 as distinct individuals in the population. The size of the population will be unchanged, but its genetic diversity will increase with the addition of new alleles 403 404 405 406.

Note that this method of introducing variation is distinctive to EPE. Other genetic processes exist for evolutionary design, such as natural selection, artificial selection, and evolutionary computation. In these variation is introduced by random mutation, effected either by nature or by machine. In EPE, mutation must be effected more or less directly by human creativity. Reliance instead on typical random mutation would lead to an accumulation of nonsense alleles in the population, which would quickly wear out the patience of human participants, and reduce participation levels below what is required for the effective selection, replication, and recombination of alleles, as described further below.

Mutation by human agency and reverse transcription in EPE is similar to reverse genetic engineering, as employed to create artificial ‘designer’ organisms. The phenogenetic engineer or designer, like the genetic engineer, works in reverse direction: from desired phenotypic traits, to the genetic encoding that would normally express them. New genetic material is then fabricated and inserted or substituted into the genotype of a target individual. The distinguishing characteristic of EPE, however, is that human agency is applied not only during the subprocess of mutation, but also during the accompanying subprocess of recombination; and it is therefore fundamental to the overall process that most (if not all) alleles be created by human agency, purposely for evaluation by human agency.

### Recombination

This procedure begins with the designer critically examining a particular gene with respect to the choice of alleles published for it. It begins with the step of selection, in which the designer initiates a search for the different alleles in the population, compares them with each other, and decides which is the best fit for his or her associated genotype.

During selection, it is necessary that the designer focus at the genetic level. For an artifact of a typically substantial size, existing in a typically numerous population of variants, it is tedious and time consuming to compare and contrast among each variant in its entirety. Instead, the designer focuses on a single gene, or cluster of genes, and examines the range of alleles which exist for it in the broader gene pool. Each candidate allele is evaluated in the full context of the target artifact associated with the designer (with which the designer is most familiar) rather than that of its source.

This is not to say that the source context is completely ignored. It will often prove useful, for example, to view an allele together with adjacent genes as they appear in the source, in order to properly evaluate the allele. The point is that an individual allele, or possibly a cluster of them, or some other fragment of the genotype, is selected-not the whole genotype as in most other evolutionary processes.

The genetic search prior to selection is not restricted to the purpose of revealing a list of different alleles. Additional information embedded in, derived from, or associated with the genetic search space may also be revealed. Examples of embedded information include commentary concerning a particular allele, or criticism of it, encoded directly in the allele (or encoded elsewhere in the genotype, with reference to the allele). Examples of derived information include parameters or statistics such as instance frequencies of different alleles; or cladistic analysis of populations and species. Examples of associated information include commentary, criticism or commercial advertising referencing a particular gene or allele in the search space, which is nevertheless published outside of that space. Such various kinds of additional information may serve useful purposes for specific embodiments of EPE, but they are not essential to EPE itself.

When a new allele is selected, the procedure continues with its replication. In this step, the new allele's genetic composition is copied from its source on the network to the designer's own local node or workspace, to form a separate instance of the new allele. Finally, substitution introduces the instance of the new allele into the genotype, replacing the instance of the old allele of the same gene, and thus altering the genotype of the individual artifact.

To continue the lexicographic example: imagine that a few days after publishing the genotype 400 of the original dictionary, A were to browse the population network in search of new alleles. Examining her <entry>gene 409, she would notice in the population the ‘a1c1 ’ allele 406 which expands the entry to 3 meanings. She selects this allele, agreeing it is an improvement over her own single meaning variant 409. She then examines the original genes 410 411 412 one by one. She selects <word>allele ‘a1c1’ 404 and <pronunciation>allele ‘a1b1’ 403. She rejects the <meaning>allele ‘a1c1’ 405, and makes an alternate change instead, thus introducing a new allele 506 of her own.

As she selects the source alleles 403 404 406 from the population, they are automatically replicated from their source genotypes 501 502 and recombined 503 504 505 into her own genotype 500. The resulting genotype might appear as follows:

<?xml version=“1.0” standalone=“no”?>
<!DOCTYPE lexicon SYSTEM “lexicon.dtd”>
<lexicon id=“a109”>

<entry id=“a1c1”>

<word id=“a1c1”>evo′lution</word>
<pronunciation id=“a1b1”>i:v [symbol] ′lu: ∫( [symbol] )n. ′εvalu:∫( [symbol] )n</pronunciation>
<meaning id=“a1a1”>

gradual development
</meaning>

<meaning id=“c1”>

a process of development and origin of species from previous forms
</meaning>

<meaning id=“c2”>

the progression of events etc. in due course
<example>the evolution of the plot</example>
</meaning>

</entry>

</lexicon>

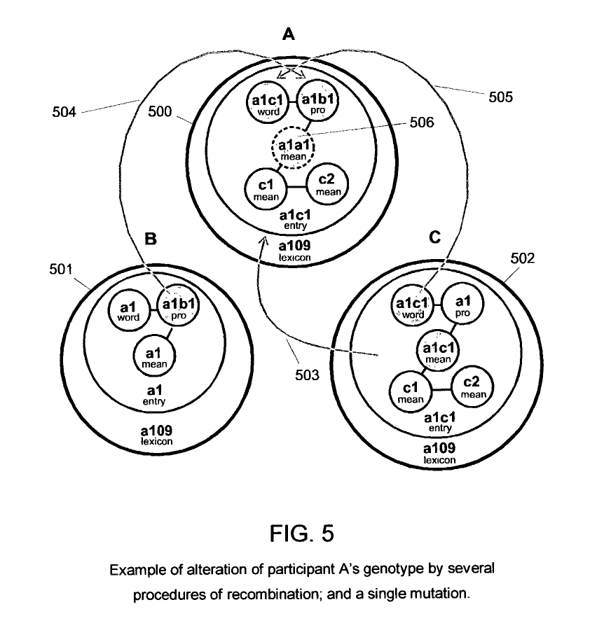

The result of these several recombinations 503 504 505 (and one mutation 506) is then republished, and the population appears as shown in FIG. 5. The population size remains at 3 individuals, represented by 3 genotypes 500 501 502. The genotype as originally published 400 has disappeared, replaced by the recombination variant 500 from A (and also by mutants 501 502 from B and C previously). Furthermore, a number of innovative alleles 403 404 406 have reproduced themselves 503 504 505 at the expense of others 409 410 411 in the gene pool of the population. And the phenotypes of the individual artifacts have improved (at least according to the opinions of A, B and C). At this point A's associated phenotype might be expressed as follows:

evo′lution i:v [symbol] ′lu:∫( [symbol] )n, ′εv-1 gradual development. 2 a process of development and origin of species from previous forms. 3 the progression of events etc. in due course (the evolution of the plot).

The essential step in the procedure of recombination is the human guided one of selection, while the steps of replication, substitution and publication are simple data manipulations that can easily be automated.

For each gene in the genome of the species, there exists a number of alleles, separate from that of other genes. There may be any number of such different alleles, from 1 to N; where N is the current size of the population of individuals. Each individual will have a single instance of one of these alleles incorporated in its own genotype. The total number of instances of alleles for any one gene will therefore be equal to N.

Where two separate instances of an allele of the same gene, in two separate individuals, have the same data content, then they are instances of a common allele uniquely defined by that data content. Each allele is represented in the population by some number of these identical instances, and together they comprise the sub-population of that allele. The number of sub-populations is equal to the number of alleles, and the combined size of all sub-populations is N.

During recombination, an instance of one allele is replaced by an instance of another allele. As a result, the sub-population of the one allele shrinks by 1, and the sub-population of the other grows by 1. If the sub-population of any allele is reduced to zero, then that allele is destroyed and lost forever (barring a mutation that recreates it).

Allele linkage may be substituted for allele replication. In this case, some form of data link is recombined into the genotype, rather than a full instance of the allele. The link points to a shared instance of the allele, e.g. on another node of the network. Typically this shared instance would be outside of the sub-population of the allele, and not encoded within any normal genotype, and thus not subject to routine alterations. Provided the link can adequately be maintained, and provided the logical effect on the process is the same, then allele linkage may prove useful if alleles are very large, or otherwise expensive to store.

Selection may also be used for purposes of mutation, rather than for recombination. In this case, genes or genetic fragments are selected from the gene pool of the population, or from other populations, or from other species. These are replicated and the replicas inserted into the target genotype, typically without retaining their original genetic identity, and typically without replacing material of that identity in the target. This is an act of mutation by definition, because it alters a genotype by means other than recombination. (It is not a recombination because it inserts the genetic material without replacing material already present; or because it replaces only a portion of an instance of an allele; or because the identities of source and target genes differ; or because there is some other difference that distinguishes it from recombination as defined.)

Mutation may be intermixed freely with the recombinatorial steps of selection, replication and substitution. The designer might choose to simultaneously mutate replicated alleles prior to substitution, or to simultaneously mutate the target genotype. This intermixing of the steps of mutation with those of recombination is logically, and in result, equivalent to a process in which they are kept separate.

Progressive artifact evolution will require successive rounds of human guided mutation and recombination. The two procedures are repeated in sequence to as the two principal subprocesses of EPE. Conceptually separate, in practice these two subprocesses are highly inter-twined—the results of mutation feeding raw material for recombination; and the results of recombination suggesting and encouraging new mutations.

With additional rounds of mutation and recombination, further improvements to the example population may be expected. Of course, for a new population of dictionaries, the most important mutations will be those which add new <entry>genes. Such mutations will expand the coverage of words.

For example, C might introduce a mutation to define the word ‘dictionary’. Afterwards, C's associated genotype might appear as follows:

<?xml version=“1.0” standalone=“no”?>
<!DOCTYPE lexicon SYSTEM “lexicon.dtd”>
<lexicon id=“a109c1”>

<entry id=“c1”>

<word id=“c1”>dic′tionary</word>
<pronunciation id=“c1”>′d [symbol] k∫( [symbol] )n( [symbol] )ri</pronunciation>
<meaning id=“c3”>

a compendium that lists and defines the words of a language
</meaning>

<meaning id=“c4”>

a reference compendium on any topic, with entries in alphabetical order
<example>dictionary of music</example>
</meaning>

</entry>

<entry id=“a1c1”>

<word id=“a1c1”>evo′lution</word>
<pronunciation id=“a1”>i:v [symbol] ′lu:∫( [symbol] )n</pronunciation>
<meaning id=“a1c1”>

gradual progressive change
</meaning>

<meaning id=“c1”>

a process of development and origin of species from previous forms
</meaning>

<meaning id=“c2”>

the progression of events etc. in due course
<example>the evolution of the plot</example>
</meaning>

</entry>
</lexicon>

And at this point, C's associated phenotype might be expressed as follows:

dic′tionary ′dIk∫( [symbol] )n( [symbol] )ri 1 a compendium that lists and defines words. 2 a reference compendium on any topic, with entries in alphabetical order (dictionary of music).

evolution i:v [symbol] lu.∫( [symbol] )n 1 gradual progressive change. 2 a process of development and origin of species from previous forms. 3 the progression of events etc. in due course (the evolution of the plot).

Although participants retain control over the progressive change of their own associated artifacts, the development of the species as a whole is not typically guided by any prescribed goal. The results may be unexpected. Certainly a population will often split into sub-populations that diverge phenotypically from each other, occasionally far enough for the establishment of a new subspecies or species. Taking such divergences into account, and encouraging them, typical embodiments of EPE will allow phenotypic engineers to restrict allele searches to within specified sub-populations, when desired.

Publication of an entire genotype is not always required. If a subset of the genotype can usefully be employed by other participants—in particular for the procedure of recombination—then its publication may help conserve system resources, especially if the genome is very large. This approach will work best for embodiments in which the expressed form and function of the species is sufficiently segmented or loosely composed (at some level) so that isolated portions of an individual are useful in themselves.

For example, in a lexicographical embodiment, a participant specialized in the vocabulary of a particular field-such as music, or astronomy-might publish a partial genotype corresponding to the terminology of that field.

Although selection is based on the criteria of expressed (phenotypic) traits, the resulting differential reproduction occurs at the genetic level, through the direct replication of instances of alleles. This is unlike biological selection, in which variants of genes and their attendant phenotypic traits are replicated primarily by differential reproduction of the larger individuals which exhibit them. Instead, in EPE, instances of alleles may replicate independently within the population, so that any theoretical recombination can occur within a single generation. This raises the potential rate of evolution; a potential which can only be realized, in fact, by the direct human guidance provided at the genetic level during mutation and selection.

Differential reproduction may still occur at the larger individual level, particularly through individual replication as described previously; but this is not essential to the process. Individual replication is only needed to enlarge the population when necessary, e.g. when new participants wish to join the process. Owing to this, selection may nevertheless occur at the individual level, as newcomers choose their favourite variants within the existing population; but on its own this would be insufficient to maintain a high rate of evolution. The innovation of direct selection at the genetic level is essential to EPE.

It will be appreciated that the above description relates to the preferred embodiments of the invention by way of the essential method only; with specific examples provided for illustration. Many variations on the method will be clear to those knowledgeable in the field, and such variations are within the scope of the invention as described and claimed, whether or not expressly described.

### System Description

Typical system embodiments of the invention will rely on data communications networks, such as the Internet, together with computer workstations and specialized software in support of EPE.

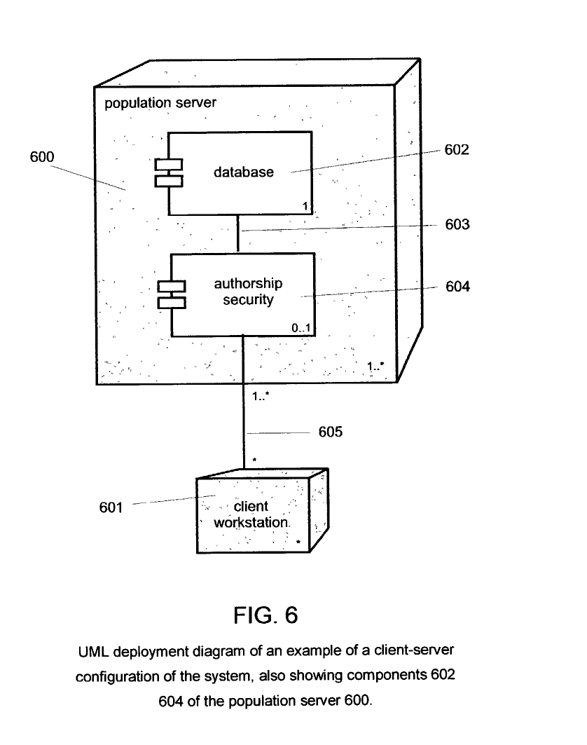

Communications might be implemented in a client-server, or alternatively in a peer-to-peer configuration. In a client-server configuration, as shown in FIG. 6, one or more dedicated population servers 600 store genotypic data for participants at remote client workstations 601. At each client workstation 601, software tools allow participants to engage in the process of EPE. Each participant works with a temporary local copy of his or her own associated genotype, or a portion of it, altering it by the procedures of mutation and recombination. The resulting altered genotype is then republished by copying its data back to the population server 600.

A population server 600 is essentially a database 602 with a secure communication interface 603 onto the network 605. Typical commercial database products are sufficient in themselves to build a working population server 600.

More advanced embodiments might interpose a layer of software between the database 602 and the network 605, in order to provide additional capabilities. An example would be a component 604 for authorship security, added to ensure that the authorship encoding of mutations could not be tampered with. This component 604 would check genotypes every time they are published to the population server 600, in order to detect unauthorized alterations. Thus authorship data encoded in the genotype could not be altered; only, for example, append to.

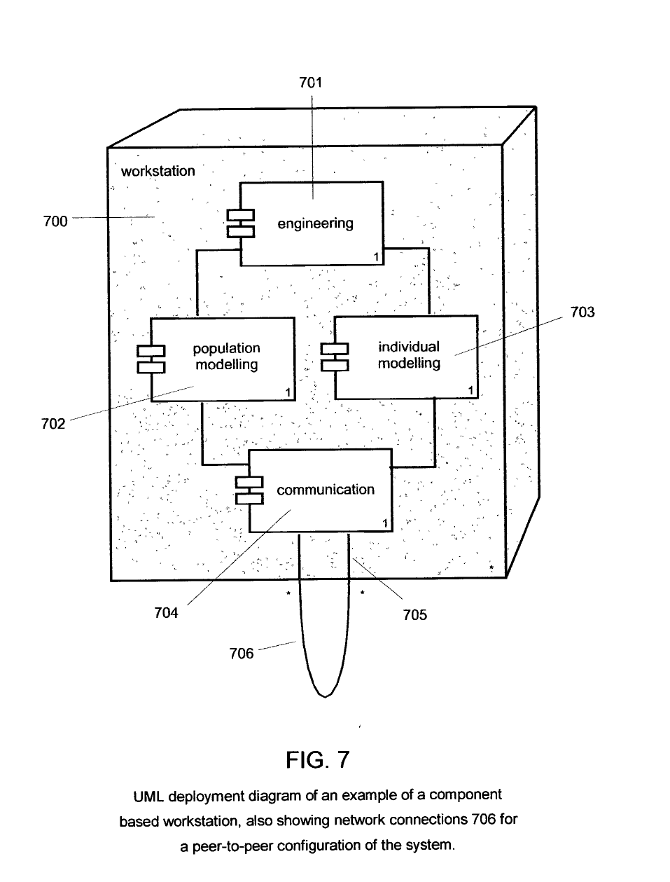

In a peer-to-peer configuration, on the other hand, there are no population servers 600. As shown in FIG. 7, each participant's workstation 700 must be able to publish the associated genotype on its own; storing a permanent copy for that purpose, and serving genotypic data to other workstations 700 on request.

Workstation 700 software may be monolithic or component based. A monolithic application is deployed as a single piece of software; whereas a component-based application is composed of separately deployable and interchangeable software parts. The following describes a component-based example. In this particular example, four different components of four different types, together implement EPE on a participant's workstation. The component types are:

Communication Component 704

Population Modelling Component 702

Individual Modelling Component 703

Engineering Component 701

Associations among these component types are shown in FIG. 7, which also shows network connections 706 for a peer-to-peer configuration of the system. (A client-server version of FIG. 7 would differ only in that the network connections 706, instead of linking workstations to workstations, would link workstations to population servers, exactly like the connections 605 of FIG. 6.)

The Communication Component 704 provides a low level interface to the populations in the form of data communication facilities for the use of other components 702 703. The communication component 704 is restricted to maintaining network connections 705, and to transferring raw data back and forth; it does not look into the genetic structure of the data, and is not concerned with the higher level process of EPE.

In a client-server configuration, the Communication Component 704 is closely matched with the communication interface 603 of the population server 600. The Communication Component 704 might be provided, in this case, by the database vendor.

In a peer-to-peer configuration, the Communication Component 704 communicates directly with other workstations 700—its peers-via the network 706. The software to implement this capability might be based on one of the newly emerging general-purpose peer-to-peer application platforms, such as Sun Microsystems' JXTA, or it might be designed from scratch by an Internet software architect.

The Population Modelling Component 702 is responsible for representing the populations within the context of the participant's workstation 700. It is used by the Engineering Component 701, and it in turn uses the Communication Component 704. One of its purposes is to conduct searches through the population for alleles of a particular gene. Each resulting list of alleles may be filtered and sorted according to specified criteria, such as source, content, lineage etc.

In peer-to-peer configurations, complex allele searches may be conducted by software agents, different types of which may be specialized for different types of searches. Such agents will be sent out by the Population Modelling Component 702, and received by the Individual Modelling Components 703 of peer workstations 700. They will interact closely and efficiently with each Individual Modelling Component 703, using the relatively fast data communications capabilities of a single node 700, prior to reporting the results of each search back to the Population Modelling Component 702, via the relatively slow network 706.

The Individual Modelling Component 703 is responsible for representing the participant's associated artifacts to their respective populations. It implements the publication of genotypes, for example, by using the facilities of the Communication Component 704.

In practice, although a participant is likely to maintain several genotypes for the same artifact, typically only a single one would be published, thus contributing to the population. The remainder will be held in local storage for reference, either as historical drafts, or as interesting variants for future consideration. A participant might also be allowed to publish multiple genotypes into the same population; effectively acting as multiple participants by doing so. Whether or not this is supported will depend on the implementation of the Individual Modelling Component 703.

In a peer-to-peer configuration, the Individual Modelling Component 703 may also provide security for authorship encodings. One method is to use nested public key encryption. In this method, the private key of the participant authoring the mutation is employed to encrypt the data of each mutation, together with the author's identity, and a mutation timestamp. This locks together all three, rendering them tamper proof, and authenticating the identity of the author. Further progressive mutations by other authors may be added in the same manner, wrapping and encrypting their predecessors.

Each author's public key is appended to the genotype, allowing the encrypted data to be read.

To guard against original mutations being copied without their associated authorship encodings, e.g. manually, the software may carry out background searches for identical mutations, and force priority to those with earlier timestamps. This requires enforced synchronization of timestamp clocks across the network, which may be implemented by cooperation among Individual Modelling Components 703; either in concert with each other, and by statistical averaging, with elimination of outliers; or by reference to a standard central time service, e.g. on the Internet.

Another method of securing authorship data is to use central encryption servers for the controlled administration of public key encryption. In this method, the private key of the server is used to encrypt the data, locked together with an official timestamp obtained from the server's clock.

The Engineering Component 701 provides an interface for the participant. Typically it will be implemented as a graphical user interface, with constructs designed to allow the participant to control the various procedures and steps of EPE.

Instances of all four component types 701 702 703 704 may be developed using modern programming languages and platforms. For example, the Java™ programming language and the Java 2 Enterprise Edition platform would be adequate.

### Example Phenogenetic Grammar in ASN.1

The following is the syntax of an example phenogenetic grammar, specified in ASN.1 (Abstract Syntax Notation One, CCITT X.208, ISO 8824).

| | |
|---|---|
| -- | ========================================================================= |
| -- | Phenogenetic syntax in ASN.1 notation. |
| -- | |

PhenogeneticSyntax DEFINITIONS ::= BEGIN

| | |
|---|---|
| -- | = = = = = = = = = = = = = = = = = = = = = = = = = = = = = = = = = = = = = |

| -- | AlleleDataSet. |
|---|---|
| -- | A set of data associated with each allele. |
| -- | |
| | AlleleDataSet ::= SET |
| | { |

locus GeneralString( SIZE( 1 . . . MAX )),
claimReference SEQUENCE SIZE(2 . . . MAX) OF CHOICE
{

SET { claimant ClaimantIndex, claimTime Time },
NullClaim

}

}
ClaimantIndex ::= SET -- compound index to ClaimantTable
{

birthDate Time,
subIndex Integer( 0 . . . MAX

}
-- - - - - - - - - - - - - - - - - - - - - - - - - - - - -
NullClaim ::= ENUMERATED { endOfSequence(1), unknown(2) }
Time ::= Integer -- offset from January 1, 1970, 00:00:00 Greenwich Mean
-- Time, in positive or negative seconds (UTC)
URI ::= GeneralString -- format per IETF RFC 2396

| | |
|---|---|
| -- | = = = = = = = = = = = = = = = = = = = = = = = = = = = = = = = = = = = = = |

| -- | ClaimantTable. |
|---|---|
| -- | The set of all claimants from across all alleles of the genotype. |
| -- | |
| | ClaimantTable ::= SET OF SET |
| | { |

index ClaimantIndex,
identification CHOICE
{

name URL, -- of claimant having no claims publisher, or
claimsPublisherAssignment ClaimsPublisherAssignment

}

}
ClaimsPublisherAssignment ::= SET
{

assignmentTime Time,
publisherSelection SEQUENCE SIZE(1 . . . MAX)

-- order by claimant preference

OF SET
{

locator URI, -- normally a URL
identification OPTIONAL INTEGER(0 . . . MAX)

-- index to identificationArray

},
identificationArray SEQUENCE OF SET
{

method URI,
tag ANY -- format per method

}

}

| | |
|---|---|
| -- | = = = = = = = = = = = = = = = = = = = = = = = = = = = = = = = = = = = = = |

| -- | GametangiumList. |
|---|---|
| -- | Known sources of gametes for this individual artifact. |
| -- | (Per individual, uncloneable.) |
| -- | |
| | Gametangiumlist ::= SET OF URI |

END

| | |
|---|---|
| -- | ========================================================================= |

The AlleleDataSet construct defines a data set associated with each allele. Any definite portion of an individual artifact's encoding that includes its own values for these data thereby qualifies as an instance of an allele, and a gene in the individual's genotype.

The locus of the AlleleDataSet identifies which gene the allele is a variant of. The locus is unique within its genotype. No two genes of the same individual share the same locus.

The phenogenetic locus is similar to its organic counterpart in that it identifies a gene, and is shared in common among the gene's alleles. It differs in being encoded directly into the gene, rather than being derived from its location in the genotype, as is the locus of an organic gene. A phenogene may therefore move within the genotype without affecting its identity. It may not, however, be duplicated within a single genotype, unless one of the duplicates takes on a new locus.

The format of the locus value is not important. It is not specified here, except to say that the default value assigned when a new gene is created should have a high probability of being unique among the genes of all individuals, and ideally of all species.

For example, a default value formed from the name and birth date of the gene's creator, plus the creation time of the gene itself, would have a high probability of being unique.

In the event that a new gene is assigned an existing locus, it will instead become an allele of the existing gene.

The locus of an instance of a gene may be changed at any time, either to a new value, or to that of another gene. At the same time, the content may also be changed, either by copying the content of another allele or gene, or by creating new content. In a hierarchically structured genotype, the restructuring that accompanies a change in locus for a particular instance of a nested subgene is a mutation of the supergene that contains it.

The claimReference of the AlleleDataSet is a sequence of at least 2 claims, that of the most recent mutation, and of its predecessor. This minimal duplication is intended to guard against weak links in the chain of authorship claims. The order of the claimReference sequence should follow the order in which the claims link to each other in the claimant's databases. This may contradict the sequence of claimTimes.

The birthDate of the claimantindex of the AlleleDataSet is not sufficient as a unique index to a claimant, and therefore the subindex is used to ensure a locally unique index to the claimantTable, valid within a single genotype. The birthDate is naturally immutable. A claimant may not assume a new birthDate.

If the size of the claimReference sequence extends beyond the original allele of the gene, then the first claimReference beyond will be a Nullclaim of value endOfSequence. Any claimReferences after endOfSequence are undefined, and should be ignored.

The claimantTable construct of the genotype is the set of all claimants from across all alleles of the genotype. It is separate from the AlleleDataSet because it is expected that different alleles will often reference the same claimant. Rather than repeat the claimant's data in each allele, they are entered once only in the claimantTable, and referenced from different alleles.

A claimant may be identified within a claimantTable record by a plain name in URI format. This method of identification is used if the claimant has no claims publisher. A plain name may be used, for example, if the claimant is an historical source. It may also be used if the claimant has not yet set up a claims database, perhaps because the claimed mutations themselves have not yet been published. The claimant name need not be a formal URN. It may be any URI.

A claimant may instead have fully published claims. In this case, the claims are located on the network as specified by the claimsPublisherAssignment of the claimantTable record.

The method of the identificationArray may be used at runtime to match the publisher's locator with the identity of a claimant, authenticating it as the true source of the claimant's claims database. The details of specific methods and the contingent format of the tag will vary from system to system. An example would be a particular algorithm of public key encryption, with the key stored in the tag.

Authentication as described here does not go beyond identification of the claimant. In particular, no method is provided for verifying the claims themselves. It is possible that claimants will make erroneous or false claims. The methods for detecting erroneous or false claims are not defined here.

The GametangiumList construct of the genotype is a list of fixed sources for published gametes of the genotype. A gamete is a copy of a genotype formed for the purpose of recombining it with another. The designer typically works with the genotype of a single artifact under design, and uses the gametes of other genotypes for recombination. The GametangiumList of each gamete may be used to obtain a up-to-date copy of the gamete, as specified by the URI.

The GametangiumList may be empty. An individual artifact may in fact have no fixed source of gametes. In this case, changes to gametes may only be discovered dynamically, e.g. using networked software such as a population modelling component 702.

The GametangiumList is unique to an individual artifact. It should not be replicated in clones. No two distinct individuals can share the same gamete sources.

Logically, gamete sources would not be part of the genotype. They are nevertheless included as a convenience for publishers to advertise sources, and for searchers to update old gametes. For these purposes it is convenient if the sources are encoded in the same file, along with the genotype of the gamete or individual, as the GametangiumList allows.

The foregoing example is incomplete, because there is no concrete language for ASN. 1 syntax, and no obvious application. The following section extends the grammar into the concrete language of XML, and completes the example.

### Example of a Phenogenetic Grammar in XML

The following is the syntax of an example phenogenetic grammar specified in DTD notation for XML. This is an XML mapping of the abstract syntax defined in the preceding section. Using this syntax, an existing XML grammar may be transformed into a phenogenetic grammar—and an existing XML document into the genotype of a phenogenetic artifact—by adding the constructs it specifies.

<!-- = = = = = = = = = = = = = = = = = = = = = = = = = = = = = = = = = = = =

| | |
|---|---|
| ! | Phenogenetic syntax in DTD notation for XML version 1.0. |

!-->

<!-- - - - - - - - - - - - - - - - - - - - - - - - - - - -

| | |
|---|---|
| ! | Namespace attribute. |
| ! | Include in the definition of the document element. |

!-->

<!ENTITY % PGE_namespace_attribute

‘xmlns:genetic CDATA #FIXED “http://www.example.com/example”’
>

<!-- - - - - - - - - - - - - - - - - - - - - - - - - - - -

| | |
|---|---|
| ! | Genotype database. |
| ! | Collects together all genotype data |
| ! | that is stored outside of the design structure. |
| ! | A single genotypeDatabase element is placed |
| ! | anywhere within the document structure; |
| ! | but preferably as a child of the document element. |

!-->

<!ELEMENT genetic: genotypeDatabase

(genetic:claimantTable, genetic:gametangiumList?)>

<!ATTLIST genetic:genotypeDatabase xmlns

CDATA #FIXED ‘http://www.zelea.com/product/EDGE/2002/PGE’>

<!-- = = = = = = = = = = = = = = = = = = = = = = = = = = = = = = = = = = = =

| | |
|---|---|
| ! | AlleleDataSet. |
| ! | A set of data associated with each allele. |

!-->

<!ENTITY % EDGE_AlleleDataSet

‘genetic:locus CDATA #IMPLIED

genetic:claimantBirthDate NMTOKEN #IMPLIED
genetic:claimantSubIndex NMTOKEN #IMPLIED
genetic: claimTime NMTOKEN #IMPLIED’
>

<!-- = = = = = = = = = = = = = = = = = = = = = = = = = = = = = = = = = = = =

| | |
|---|---|
| ! | ClaimantTable. |
| ! | The set of all claimants from across all alleles of the genotype. |

!-->

<!ELEMENT genetic:claimantTable (genetic:claimant*)>
<!ELEMENT genetic:claimant

(genetic:name|genetic:claimsPublisherAssignment)>

<!ATTLIST genetic:claimant genetic:birthDate NMTOKEN #REQUIRED>
<!ATTLIST genetic:claimant genetic:subIndex NMTOKEN #REQUIRED>

<!ELEMENT genetic:name EMPTY>

<!ATTLIST genetic:name genetic:uri CDATA #REQUIRED>

<!ELEMENT genetic:claimsPublisherAssignment

(genetic:publisherSelection,genetic:identificationArray)>

<!ATTLIST genetic:claimsPublisherAssignment

genetic:assignmentTime NMTOKEN #REQUIRED>

<!ELEMENT genetic:publisherSelection (genetic:publisher+)>
<!ELEMENT genetic:publisher EMPTY>

<!ATTLIST genetic:publisher genetic:locator CDATA #REQUIRED>
<!ATTLIST genetic:publisher genetic:identification NMTOKEN #IMPLIED>

<!ELEMENT genetic:identificationArray (genetic:identification*)>
<!ELEMENT genetic:identification (genetic:tag)>

<!ATTLIST genetic:identification genetic:method CDATA #REQUIRED>

<!ELEMENT genetic:tag ANY>

<!-- = = = = = = = = = = = = = = = = = = = = = = = = = = = = = = = = = = = =

| | |
|---|---|
| ! | GametangiumList. |
| ! | Known sources of gametes for this individual artifact. |
| ! | (Per individual, uncloneable.) |

!-->

<!ELEMENT genetic:gametangiumList (genetic:gametangium*)>
<!ELEMENT genetic:gametangium EMPTY>

<!ATTLIST genetic:gametangium genetic:uri CDATA #REQUIRED>

Phenogenetic elements and attributes are prefixed with ‘genetic:’ which, in this example, is associated with the fictitious namespace ‘http://www.example.com/example’. The prefix ‘genetic:’ is used by convention to denote the namespace, although any prefix can be used. The purpose of the association is to avoid conflict between the phenogenetic extensions and any other extensions to the grammar that might be specified for the application.

Any future version of the phenogenetic extensions that might break existing applications would be associated with a different namespace.

The locus of the document element at the root of the hierarchical genotype identifies the genome and hence the species of the individual. This would typically limit the populations it could join for purposes of recombination. A species, by this definition, is the set of all individuals that share a document element of the same locus.

This example grammar meets the requirements of a phenogenetic grammar as specified previously, except for requirement number 8, as explained below.

1) It specifies that genes and their data contents are encoded as XML elements.

2) It specifies that a genotype is composed as an XML document.

3) It allows for an individual artifact's phenotype to be expressed from its genotype as a transformation or interpretation of an XML document. The detailed mechanism, however, would depend on the exact application. E.g., if the document type is XHTML (Extensible Hyper-Text Markup Language) then the document would be expressed by viewing it in a web browser, or by printing it, etc.

4) It allows for the encoding of a wide range of genotypes, owing to the flexibility and extensibility of XML. The exact range, however, would depend on the application.

5) It enables individual artifacts to be replicated as XML documents, using ordinary data copying.

6) It enables genotypes to be mutated using an XML editor/viewer; either a general purpose one, or one that is specialized for the application.

7) It enables genotypes to be recombined using standard XML Fragment Interchange. During recombination, transferred alleles are each given appropriate entries in the claimantTable of the target genotype, and their genetic:claimantsubindex attribute is adjusted to match.

8) It does not, however, define a mapping of genotypes to phenotypes in such a way as to facilitate human guided mutation and selection of alleles, according to their phenotypic traits. This would depend entirely on the application. For example, in a text application of the XHTML document type, each paragraph gene encoded as<p>element would clearly and directly correspond to a readable paragraph when rendered in a web browser; and each line, phrase or sentence gene encoded as a <div>or <span>element would correspond to a line, phrase or sentence when rendered in a web browser; and so on for all of the element types one might wish to encode as genes.

9) It specifies that the unique identity of each gene is encoded as a genetic:locus attribute in each of its allele instances.

10) It specifies that the authorship of mutations is encoded using genetic:claimantBirthDate and genetic:claimantsubindex attributes of an allele together as a compound index into the claimantTable construct of the genotype. The claimantTable in turn provides the identity of the claimant. This, together with the allele's genetic:claimTime attribute, is sufficient to establish a specific claim of authorship with regard to the mutation that created the allele. Obtaining the authorship of prior mutations to establish the full authorship history of an allele would require establishing contact with the claims database of the author of the latest mutation, or the one prior to that, etc. Details of how this is done, and how a claims database would be implemented are not provided here, as they are not essential to the invention.

It will be appreciated that the system and phenogenetic grammars described above relate to the preferred embodiments of the invention by way of example only. Many variations on the apparatus for delivering the invention will be clear to those knowledgeable in the field, and such variations are within the scope of the invention as described and claimed, whether or not expressly described.

### Glossary

allele a specific variant of a gene defined by its unique data content.

EPE evolutionary phenogenetic engineering.

express to generate a phenotype from a genotype.

gene a data structural unit of a genome, formed according to a phenogenetic grammar, that expresses a particular phenotypic trait.

gene pool the collected alleles of a population or species.

genome the abstract genotype of a species defined by a particular set of genes, and combined together according to a phenogenetic grammar.

genotype a concrete instance of a genome formed from a particular set of alleles, and sufficient to express the phenotype of an individual.

individual a single design with its own instance of a genotype and phenotype.

mutation any alteration to a genotype, except that of recombination; including changes to the content of genes, deletion or addition of genes, and other rearrangements of genes with respect to each other.

phenogenetic grammar a set of rules for encoding genetic information, in which the genotype-to-phenotype mapping, and the identity encoding of genes, are both designed to facilitate the method of EPE.

population a set of individuals of a particular species sharing a gene pool for purposes of recombination.

recombination alteration of a genotype by the substitution of one allele for another, resulting in a new genotype with a different combination of alleles.

species the universal set of all individuals of which any subset is a valid population.

### REFERENCES

Bentley, Peter (1999) An introduction to evolutionary design by computers. Evolutionary Design by Computers. Peter Bentley editor. Morgan Kaufman, San Fransisco.

Dawkins, Richard (1982) The Extended Phenotype: The Gene as the Unit of Selection. W. H. Freeman, Oxford, U.K.

Hirschberg, Urs and Florian Wenz (2000) Phase(x)-memetic engineering for architecture. Automation in Construction, 9, 387-392.

Kvan, Thomas (2000) Collaborative design: what is it? Automation in Construction, 9, 409-415.

Nagy, Gregory (1992) Introduction. The Iliad. Homer. Robert Fitzgerald translator. Alfred A. Knopf, New York.

Ong, Walter J. (1982) Orality and Literacy: the Technologizing of the Word. Methuen, London.

Parry, Milman (1928) L'Epithete traditionelle dans Homére. Doctoral thesis, Société Éditrice Les Belles Lettres, Paris. (As cited in Ong, 1982)

Raymond, E. (1997) The Cathedral and the Bazaar. http://www.tuxedo.org/˜esr/writingslcathedral-bazaar/.

I claim as my invention:

1. A participative method for facilitating the evolutionary design of a species of artifact, the species having a population consisting of individuals of the species, each individual encoded by an instance of a genotype, each genotype formed according to a phenogenetic grammar, each individual and its instance of a genotype associated with a participant from a community of participants, the community interlinked by a data network, and the method comprising the following steps:
selecting an instance of a genotype associated with a participant under direction of the participant;
applying an alteration procedure to the instance of the genotype under direction of the participant, wherein the alteration procedure comprises at least one procedure selected from the group consisting of mutations and recombinations; and
publishing via the network the result of the alteration procedure, whereby the result may be displayed to participants of the community.

2. The method of claim 1, wherein the result of the alteration procedure published is the altered portion of the genotype.

3. The method of claim 1, wherein the result of the alteration procedure published comprises the altered portion of the genotype.

4. The method of claim 1, wherein the result of the alteration procedure published comprises the entire genotype.

5. The method of any one of claims 1 to 4, wherein the mutations comprise deleting a gene from the instance of the genotype, whereby creating a new allele.

6. The method of any one of claims 1 to 4, wherein the mutations comprise altering the content of a gene being part of the instance of the genotype, whereby creating a new allele.

7. The method of any one of claims 1 to 4, wherein the mutations comprise adding a gene, the content of the gene being defined by the participant, to the instance of the genotype, whereby creating a new allele.

8. The method of any one of claims 1 to 4, wherein the mutations comprise rearranging a gene with respect to other genes within the instance of the genotype, whereby the location of the gene is modified within the instance of the genotype and creating a new allele.

9. The method of any one of claims 1 to 4, wherein the mutations comprise introducing a pre-existing gene or genetic fragment selected from the group of sources consisting of the population, other populations, and other species, into the instance of the genotype, whereby creating a new allele.

10. The method of any one of claims 1 to 4, wherein the recombinations comprise the following steps:
selecting by the participant, an allele, the allele being a published allele, and further being an allele of a gene;
replicating the allele to create a new instance of the allele; and
substituting the new instance of the allele into the instance of the genotype, wherein the new instance of the allele replaces an instance of a different allele of the gene.

11. The method of any one of claims 1 to 4, wherein the phenogenetic grammar complies with the standard prescribed under Extensible Markup Language (XML).

12. The method of any one of claims 1 to 4, wherein the phenogenetic grammar specifies a hierarchical structure for a genotype, in which a single gene may itself be composed of smaller genes nested within it.

13. The method of any of claims 1 to 12, wherein the phenotypes of the population are written language compositions.

14. The method of any of claims 1 to 12, wherein the phenotypes of the population are audio compositions.

15. The method of any of claims 1 to 12, wherein the phenotypes of the population are visual compositions.

16. The method of any of claims 1 to 12, wherein the phenotypes of the population are multi-media compositions.

17. The method of claim 13, wherein the written language compositions are creative literary compositions.

18. The method of any of claims 13 and 14, wherein the compositions are creative musical compositions.

19. The method of claim 15, wherein the visual compositions are creative graphical compositions.

20. The method of any of claims 1 to 12, wherein the phenotypes of the population are creative dramatic compositions.

21. The method of any of claims 1 to 12, wherein the phenotypes of the population are of a species chosen from the group consisting of compendia, compilations and arrangements assembled from various contributors.

22. The method of claim 21, wherein the species consists of lexicons.

23. The method of claim 21, wherein the species consists of encyclopedia.

24. The method of claim 21, wherein the species consists of travel guides.

25. The method of claim 21, wherein the species consists of cookbooks.

26. The method of any of claims 1 to 12, wherein the phenotypes of the population are translations to one language, of artifacts originally expressed in a different language.

27. The method of any of claims 1 to 12, wherein the phenotypes of the population are of a species chosen from the group consisting of industrial and commercial designs.

28. The method of claim 27, wherein the species consists of computer software.

29. The method of claim 27, wherein the species consists of integrated circuitry.

30. The method of claim 27, wherein the species consists of chemical molecular compounds.

31. The method of claim 27, wherein the species consists of biological genetic sequences.

32. The method of any of claims 1 to 12, wherein the phenotypes of the population are of a species chosen from the group consisting of rules, regulations, and laws.

33. The method of any of claims 1 to 12, wherein the phenotypes of the population are architectural designs.

34. A system for facilitating the evolutionary design of a species of artifact, the species having a population consisting of individuals of the species, each individual encoded by an instance of a genotype, each genotype formed according to a phenogenetic grammar, each individual and its instance of a genotype associated with a participant from a community of participants, the system comprising software and hardware elements forming a network of computers, said software and hardware elements comprising:
a first element for selecting an instance of a genotype associated with a participant under direction of the participant;
a second element for applying an alteration procedure to the instance of the genotype under direction of the participant, wherein the alteration procedure comprises at least one procedure selected from the group consisting of mutations and recombinations; and
a third element for publishing via the network the result of the alteration procedure, whereby the result may be displayed to participants of the community.

35. The system of claim 34, wherein the network is based on a client-server model.

36. The system of claim 34, wherein the network is based on a peer-to-peer model.

37. The system of claim 34, wherein the phenogenetic grammar complies with the standard prescribed under Extensible Markup Language (XML)
